(19) United States
(12) Patent Application Publication
(10) Pub. No.: US 2024/0293246 A1
(43) Pub. Date: Sep. 5, 2024

(54) CERVICAL COLLAR BACK PANEL

(71) Applicant: Aspen Medical Products, LLC, Irvine, CA (US)

(72) Inventors: Jane Price, Anaheim, CA (US); Erik Zimmer, Oceanside, CA (US)

(21) Appl. No.: 18/117,269

(22) Filed: Mar. 3, 2023

(51) Int. Cl. A61F 5/055

(52) U.S. Cl. CPC A61F 5/055

(57) ABSTRACT

A back panel for a cervical collar is described. The cervical collar back panel features an attachment panel and one or more cervical panels. In one embodiment, a pair of cervical panels are arranged to provide a symmetric architecture during a donning process to support multiple orientations. For example, a first cervical panel may be oriented above a second cervical panel in a first orientation or below the second cervical panel in a second orientation. In another embodiment, a single cervical panel features a first segment and a second segment. Each of these segments supports occipital and upper trapezius support on different sides of the cervical spine.

### FIELD

Embodiments of the disclosure relate to the field of orthopedic braces. More specifically, one embodiment of the disclosure relates to a collar back panel configured for attachment to a cervical collar.

### GENERAL BACKGROUND

The following description includes information that may be useful in understanding the described invention. It is not an admission that any of the information provided herein is prior art or relevant to the presently claimed invention, or that any publication specifically or implicitly referenced is prior art.

Cervical collars are used to substantially immobilize the cervical spine for treatment of a neck or cervical spine condition. These collars may assist in the treatment of whiplash, back surgery, and other similar conditions, where support for the head and neck of a wearer (hereinafter, “patient”) is needed. For some orthopedic braces, the cervical collar is merely one component of the brace, which is configured to prevent movement in a wearer's spine from the cervical region (neck and upper back region).

A cervical collar includes a front collar body and a collar back panel. Currently, the collar back panel is a single-piece component, where attachment to the cervical collar may be achieved only through a single, unique deployment scheme. Hence, conventional collar back panel architectures are not designed to reduce healthcare provider error during the donning process and, in some cases, fail to provide sufficient stabilization of the patient's neck. Conventional, cervical collar back panel architectures need to improve to mitigate the donning errors and develop a back panel architecture that finds a balance between immobilization of the cervical spine and patient comfort to ensure compliance with wearing instructions.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1 is a posterior view of a first exemplary embodiment of a cervical collar back panel.

FIG. 2A is a posterior view of an illustrative embodiment of an attachment panel of FIG. 1.

FIG. 2B is a posterior view of an illustrative embodiment of one of the cervical panels of FIG. 1.

FIG. 3A is an exploded, posterior view of the cervical collar back panel of FIG. 1.

FIG. 3B is a detailed view of the interconnection between the central panel member and one of the cervical panels of FIG. 3A.

FIG. 4 is an anterior view of the cervical collar back panel of FIGS. 1 & 3.

FIG. 5 is a posterior view of a second exemplary embodiment of a cervical collar back panel.

FIG. 6A is a posterior view of an illustrative embodiment of an attachment panel of FIG. 5.

FIG. 6B is an anterior view of an illustrative embodiment of the cervical panel of FIG. 5.

FIG. 7 is an exploded, posterior view of the cervical collar back panel of FIG. 5.

FIG. 8 is an anterior view of the cervical collar back panel of FIGS. 5 & 7.

FIG. 9 is an illustrative embodiment of a curvature of the cervical collar back panel of FIGS. 5 & 7-8.

FIG. 10 is an illustrative embodiment of a donning process in which a strap attached to a first extension member of the attachment panel is to be wrapped around a lateral side of a patient's neck for attachment to a front collar body of the cervical collar.

FIG. 11 is an illustrative embodiment of the donning process in which a central member of the attachment panel is positioned to support to the lordotic curve of the cervical spine of the patient.

FIG. 12 is an illustrative embodiment of the donning process in which strap is coupled to a portion of the front body of the cervical collar.

### DETAILED DESCRIPTION

Embodiments of the present disclosure generally relate to a cervical collar back panel, which is the posterior component for a cervical collar. According to a first embodiment of the disclosure, the cervical collar back panel features one or more cervical panels that are coupled to an attachment panel, albeit the cervical collar back panel may be constructed as a single integrated component. Herein, for this embodiment, a pair of cervical panels are designed in which one of the cervical panels is positioned to support the occipital region (e.g., occipital bone) of the patient's skull while the other cervical panel is positioned to receive support by the upper trapezius region (e.g., upper trapezius muscle area proximate to the cervical spine) of the patient. Depending on its positioning to a central member of the attachment panel, when worn, a cervical panel may feature (i) a concave shape to rest against and support the occipital region of the patient or (ii) a convex shape to rest against and be supported by the upper trapezius region of the patient.

As a result, when installed and operating as part of the cervical collar, the cervical collar back panel deploys a symmetric architecture where either cervical panel may be used to support the occipital region of the patient. Stated differently, the cervical panels are positioned with a symmetric architecture to allow one of these cervical panels to engage with the occipital region of the patient and the other to engage with the upper trapezius region of the patient.

According to this embodiment, the attachment panel features a central panel member, a first panel extension, and a second panel extension. The central panel member features a centralized aperture with periphery members surrounding the aperture. From a posterior perspective, the top/bottom periphery members are configured with a gradual concave form. In particular, the top periphery member is configured with an incline angle (e.g., upward slope) tending to veer away from an anterior side of the cervical collar back panel while the second periphery member is configured with a decline angle veering from an anterior side of the cervical collar back panel. These concave-shaped top/bottom periphery members assist in the orientation of the cervical panels to support the occipital region of the patient and be supported by the upper trapezius region of the patient. Each of the panel extensions protrudes laterally in opposite directions from the central panel member and are constructed to provide lateral support for straps that are attached to a front collar body of the cervical collar. This embodiment of the cervical collar back panel achieves a symmetric architecture by the first cervical panel being oriented in a mirrored configuration with the second cervical panel so that these cervical panels may be located at the occipital region or upper trapezius region of the patient. Stated differently, the cervical collar back panel is wearable in a first orientation (e.g., first cervical panel situated above the second cervical panel) or a second orientation rotated one-hundred and eighty degrees From the first orientation (e.g., second cervical panel situated above the first cervical panel).

According to a second embodiment of the disclosure, the cervical collar back panel features a cervical panel configured for attachment to an attachment panel, albeit the cervical collar back panel may be constructed as a single integrated component. Herein, for this embodiment, the attachment panel has a similar construction as the prior attachment panel, albeit the cervical panel is not attached to the central member of the attachment panel. Another difference rests in the architecture and function of the cervical panel, which features a first segment and a second segment. When installed and operating as part of the cervical collar, the first segment is configured to support a first lateral portion of the occipital region of the patient and receive support from a first lateral portion of the upper trapezius region of the patient. The second segment is configured to support a second lateral portion of the occipital region of the patient and receive support from a second lateral portion of the upper trapezius region of the patient. As the straps of the cervical collar back panel are attached to the front cervical collar body, these segments are compressed inwardly to provide the support noted above.

Herein, according to one embodiment of the disclosure, when the cervical collar back panel is coupled to the front cervical collar body of the cervical collar, the first and second segments of the cervical panel would be positioned on opposite sides of the cervical spine.

Herein, the cervical panel features (i) a central aperture, (ii) the first segment and (iii) the second segment. The first segment features (i) a first curved periphery member to rest against and support a corresponding lateral portion of the occipital region of the patient and (ii) a second curved periphery member to rest against and be supported by a first lateral portion of the upper trapezius region of the patient. The first segment further features a middle infrastructure that is concave-shaped from a first side edge of the central aperture to a first lateral periphery member. The second segment has a similar architecture, including (i) a third curved periphery member to rest against and support the other corresponding lateral portion of the occipital region, (ii) a fourth curved periphery member to rest against and be supported by the second lateral portion of the upper trapezius region, and a middle infrastructure that is concave-shaped from a second side edge of the central aperture to a second lateral periphery member.

As a result, when installed and operating as part of the cervical collar, the cervical collar back panel features segments that support a symmetric architecture, where the first segment is oriented laterally from and in a mirrored configuration with the second segment so that the cervical collar back panel is wearable in a first orientation (e.g., the first/second segments are located right/left of the central axis) or a second orientation rotated one-hundred and eighty degrees From the first orientation (e.g., the first/second segments are located left/right of the central axis). Stated differently, the cervical panel features segments that are positioned to allow one of these segments to engage with the first lateral portion of the occipital region of the patient and the first lateral portion of the upper trapezius region of the patient. The other one of these segments is configured to engage with the second lateral portion of the occipital region of the patient and the second lateral portion of the upper trapezius region of the patient, where the first and second lateral portions are positioned on different sides of the cervical spine.

### I. TERMINOLOGY

In the following description, certain terminology is used to describe aspects of the invention. For example, each of the terms “member” and “panel” may be construed as a physical component, where the physical component may be part of an orthopedic brace such as a cervical collar for example. In certain situations, the member (or panel) may rely on one or more fasteners (hereinafter, “fastener(s)”) for attachment of that member (or panel) to a different member (or panel) that features complementary fastener(s). For example, a first member may include a “loop” type fastener to which a “hook” type fastener located on a second member may be attached or the first member may include a hook-type fastener for attachment to a loop-type fastener located on the second member.

The term “attach” and other verb tenses of the term (attached, attaching, etc.) may be construed as physically connecting two items together. This physical connection may be a direct connection of the two items using fastener(s) or an indirect connection that relies on one or more intermediary members to which the two items are connected. The term “fastener” may be construed as any physical component used to attach different items together. An illustrative example of different types of fasteners and fastening techniques may include, but are not limited or restricted to a snap, button, clasp, clip, hook or loop element (e.g., Velcro®), buckle, adhesive, rivet, screw, bolt, heat seal (or melting), or other component that is connected to its complementary fastener.

The term “concave” may be construed as a curvature in a first direction (e.g., upward like an interior of a circle) while the term “convex” may be construed as a curvature in a second direction (e.g., downward like an exterior of a circle“). It is contemplated that these curvatures may be alterable with a change in viewpoint, where a surface may be convex from a posterior viewpoint and concave from an anterior viewpoint. As a result, for the subject application, the viewpoints for the figures are identified.

The term “rigid” with respect to a member or portion of a member may be construed as the member could be permanently deformed or broken if bent or twisted by at least 90°.

Finally, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

As this invention is susceptible to embodiments of many different forms, it is intended that the present disclosure is to be considered as an example of the principles of the invention and not intended to limit the invention to the specific embodiments shown and described.

### II. GENERAL ARCHITECTURE

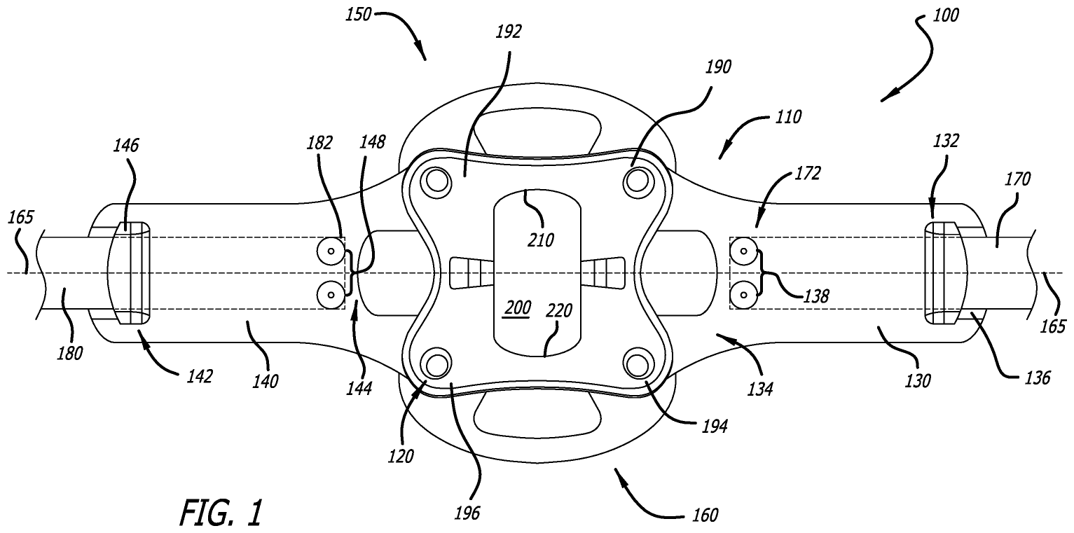

Referring to FIG. 1, a posterior view of a first embodiment of a cervical collar back panel 100 is shown. According to this embodiment, the cervical collar back panel 100 includes an attachment panel 110, a first cervical panel 150 and a second cervical panel 160. The attachment panel 110 includes a central panel member 120, a first extension member 130, and a second extension member 140. The central panel member 120 is configured to retain the first cervical panel 150 and the second cervical panel 160, which are attached together through one or more fasteners (not shown). Alternatively, the cervical collar back panel 100, inclusive of the attachment panel 110, the first cervical panel 150 and the second cervical panel 160, may be constructed as a single integrated component.

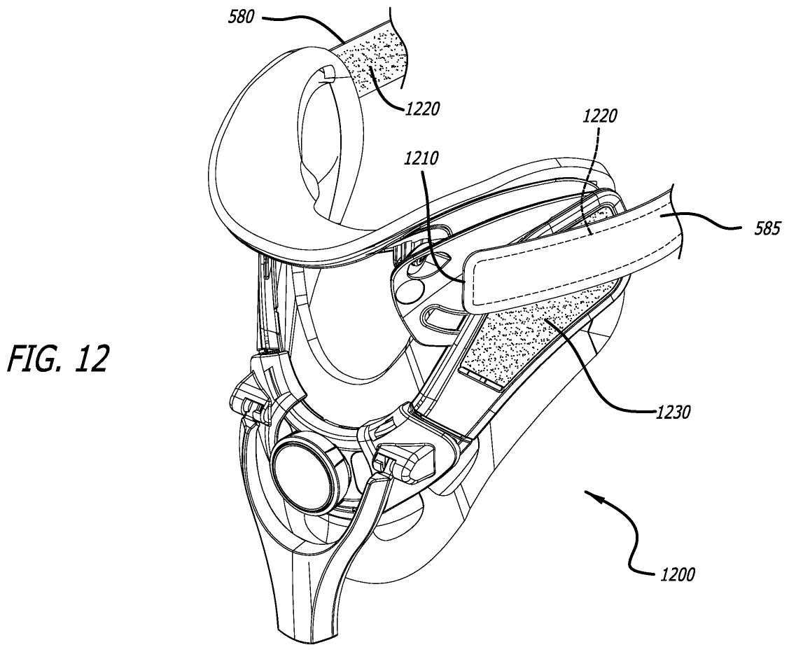

The first extension member 130 includes a distal region 132 and a proximal region 134. The proximal region 134 is coupled to the central panel member 120 while the distal region 132 features a slot 136 to receive a strap 170 that is used for connectivity to a front collar body 1200 (see FIG. 12) of a cervical collar. The first extension member 130 further includes a plurality of female fasteners 138 adapted to receive a male fastener (e.g., a rivet) for attaching an end 172 of the strap 170 to the attachment panel 110.

The second extension member 140 includes an architecture similar to the first extension member 130, where the slot 146 located in a distal region 142 of the second extension member 140. The slot 146 is configured to receive the strap 180 located on the anterior side of the second extension member 140 for re-location to the posterior side of the second extension member 140. A plurality of female fasteners 148 are located in the proximal region 144 of the second extension member 140 and adapted to receive a male fastener (e.g., a rivet) to secure an end 182 of the strap 180. Herein, the straps 170/180 are different straps to avoid interfering with the maneuverability of the extension members 130 and 140.

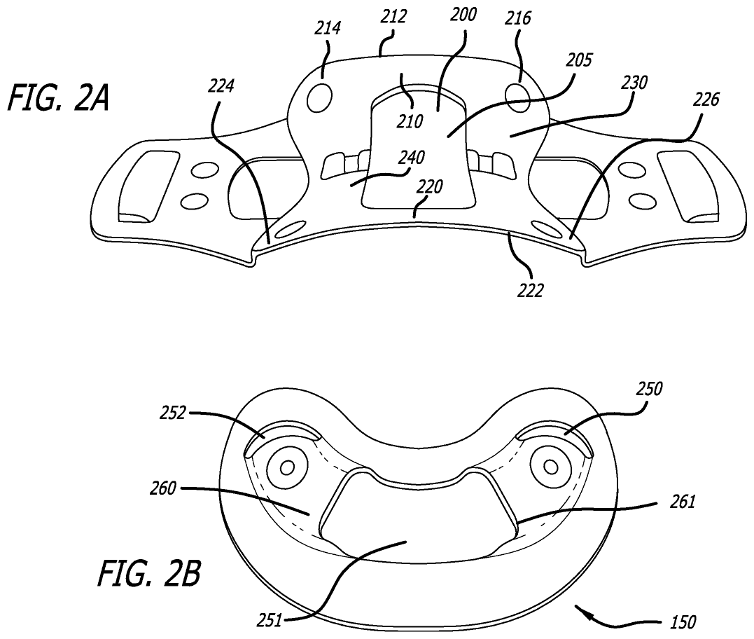

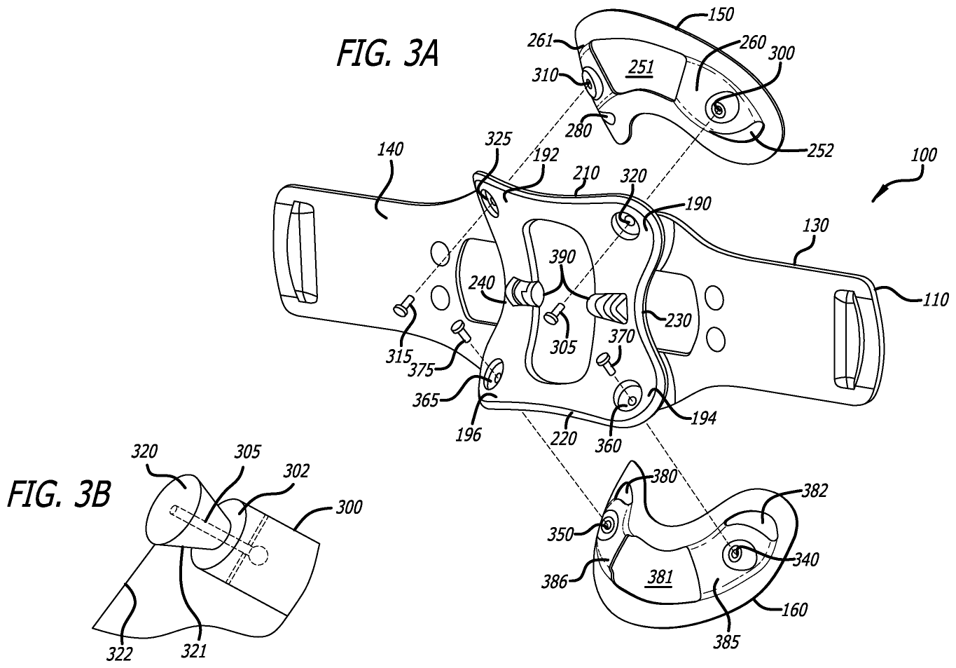

The cervical collar back panel 100 further includes the first cervical panel 150 and the second cervical panel 160, which are positioned symmetric to each other from a traverse axis 165 intersecting the central panel member 120. Both of the first and second cervical panels 150 and 160 feature a curved structure that is complementary to the curved form of the anterior surfaces of a first periphery member 210 and a second periphery member 220 of the central panel member 120 (see FIG. 2A). As shown, for a posterior viewpoint, the first cervical panel 150 features a convex configuration and is positioned at an offset distance from an anterior surface of the first periphery member 210. This offset is accomplished from recessed openings formed in the first periphery member 210 having hemispherical convex protrusions extending from the anterior surface of the first periphery member 210 and engaging with fasteners of the first cervical panel 150 as shown in FIG. 3B and described below.

The first cervical panel 150 is configured and shaped to conform to the occipital region of the patient. The first cervical panel 150 is positioned at an offset distance from an anterior surface of the first periphery member 210 to allow for articulation and attached at intersection regions 190 and 192 of the central panel member 120. Similarly, the second cervical panel 160 features a concave configuration, is positioned at an offset distance from an anterior surface of the second periphery member 220 and attached at intersection regions 194 and 196. When the cervical collar is worn, the first and second cervical panels 150 and 160 are interchangeable as both of these cervical panels 150 and 160 may be used to support the occipital region or the upper trapezius region of the patient.

Referring now to FIG. 2A, an illustrative embodiment of the attachment panel 110 of FIG. 1 is shown. According to this embodiment, the central panel member 120 features a centralized aperture 200 formed by a plurality of periphery members, including the first periphery member 210 and the second periphery member 220, as well as a third periphery member 230 and a fourth periphery member 240. Herein, from a posterior viewpoint, the first periphery member 210 is configured with a concave (e.g., upward sloping) surface from the aperture 205 to its edge 212 while the edge 212 is convex in form between its first distal end 214 and second distal end 216. This convex shape expands the offset to provide sufficient spacing to allow the first cervical panel 150 to articulate based on forces exerted on the first cervical panel 150 until contacting the anterior surface of the first periphery member 210. The second periphery member 220 is configured with a concave surface from the aperture 205 to its edge 222. The edge 222 is convex in form between its first distal end 224 and second distal end 226.

Referring still to FIG. 2A, the third periphery member 230 is configured with a concave surface from the edge 212 of the first periphery member 210 to the edge 222 of the second periphery member 220, where the concave surface is an aggregate of the concave surfaces of the first/second periphery members 210/220 from the aperture 205. Similarly, the fourth periphery member 240 is configured with the concave surface from the edge 212 of the first periphery member 210 to the edge 222 of the second periphery member 220.

In summary, with respect to the configuration of the periphery members 210, 220, 230 and 240, the first periphery member 210 is configured with an incline angle (e.g., upward slope) tending to veer away from an anterior side of the cervical collar back panel 100 while the second periphery member 220 is configured with a decline angle veering away from an anterior side of the cervical collar back panel 100. As a result, the first periphery member 210 is arranged to assist in the orientation of the first cervical panel 150 to support an occipital region of the patient and contribute to establishing an offset distance between a posterior surface of the first cervical panel 150 and an anterior surface of the first periphery member 210. The second periphery member 220 is arranged to establish an offset distance between its anterior surface and a posterior surface of the second cervical panel 160 as well as assist in the orientation of the second cervical panel 160 to rest on and be supported by an upper trapezius region of the patient.

Referring to FIG. 3A, an exploded view of the cervical collar back panel 100 of FIG. 1 is shown. The cervical collar back panel 100 includes the attachment panel 110, the first cervical panel 150, and the second cervical panel 160. As shown, the first cervical panel 150 includes a plurality of fasteners 300/310, which include a first fastener 300 and a second fastener 310. The first fastener 300 is formed as part of the cervical panel 150 for alignment with a recessed opening 320 formed within the intersection region 190 of the first/third periphery members 210/230 of the central panel member 120. The second fastener 310 is formed for alignment with a recessed opening 325 within the intersection region 192 of the first/fourth periphery members 210/240 of the central panel member 120.

Herein, as shown in FIG. 3B, the recessed opening 320 forms a hemispherical convex protrusion 321 extending from an anterior surface 322 of the first periphery member 210. The protrusion 321 is aligned with and engages with a hemispherical concave portion 302 of the fastener 300. The engagement may be accomplished where the protrusion 321 partially rests in the hemispherical concave portion 302 or the protrusion 321 abuts against the hemispherical concave portion 302.

Referring to both FIGS. 3A-3B, fasteners 305/315 are inserted into the recessed openings 320/325 and attached to fasteners 300/310 of the cervical panel 150, respectively. According to one embodiment of the disclosure, as specifically shown in FIG. 3B, a male rivet 305 may be sized with a diameter for (i) insertion through the recessed opening 320, (ii) extension beyond the protrusion 321 and (iii) insertion into the fastener 300 of the first cervical panel 150. Upon insertion, the rivet 305 is secured to the fastener 300 and is unable to be removed without significant force being applied. As a result, the first cervical panel 150 is coupled to the central panel member 120 of the attachment panel 110. This coupling, along with a construction featuring apertures 250-252 between raised, fastener regions 260-261 as shown in FIG. 2B, provide the first cervical panel 150 with flexibility to account for differences in the shape of the occipital region (e.g., occipital bone) for different patients.

With a similar construction as the first cervical panel 150, the second cervical panel 160 includes a plurality of fasteners 340/350, which include a third fastener 340 and a fourth fastener 350. The third fastener 340 is formed for alignment with a recessed opening 360 within the intersection region 194 of the second/third periphery members 220/230 of the central panel member 120. The fourth fastener 350 is formed for alignment with a recessed opening 365 within the intersection region 196 of the second/fourth periphery members 220/240 of the central panel member 120.

As further shown in FIG. 3A, fasteners 370/375 may be inserted into the recessed openings 360/365 and attached to the fasteners 340/350 of the second cervical panel 160, respectively. As a result, the second cervical panel 160 is coupled to the central panel member 120 of the attachment panel 110. This coupling, along with its construction featuring apertures 380-382 between fastener regions 385-386, provide the second cervical panel 160 with flexibility to account for differences in the shape of the upper trapezius region for different patients.

As further shown in FIG. 3A, the central panel member 120 further includes inserts 390 for attachment to a spinal stabilization rod such as a wishbone coupler of a Cervical Thoracic Lumbar Sacral Orthosis (CTLSO) described in U.S. patent application Ser. No. 18/162,367 filed Jan. 31, 2023 and entitled “Orthopedic Brace Having Telescopic Lateral Panels and an Interchangeable Wishbone Coupler,” the contents of which are incorporated by reference herein. Given the rigidity of the central panel member 120, a stabilization rod for the CTLSO may be attached to the inserts 390 and would not lose alignment as the first and second extension panel members 130 and 140 are flexed.

Herein, according to one embodiment of the disclosure, when attached to the central panel member 120, the second cervical panel 160 is oriented in a mirrored manner to the orientation of the first cervical panel 150. As a result, one of the first/second cervical panels 150/160 may be positioned to provide support for the occipital region of the patient, where the other of the first/second cervical panels 150/160 may be positioned to receive support from the upper trapezius region of the patient. This symmetric architecture mitigates errors during the donning of the cervical collar by a healthcare provider or the patient.

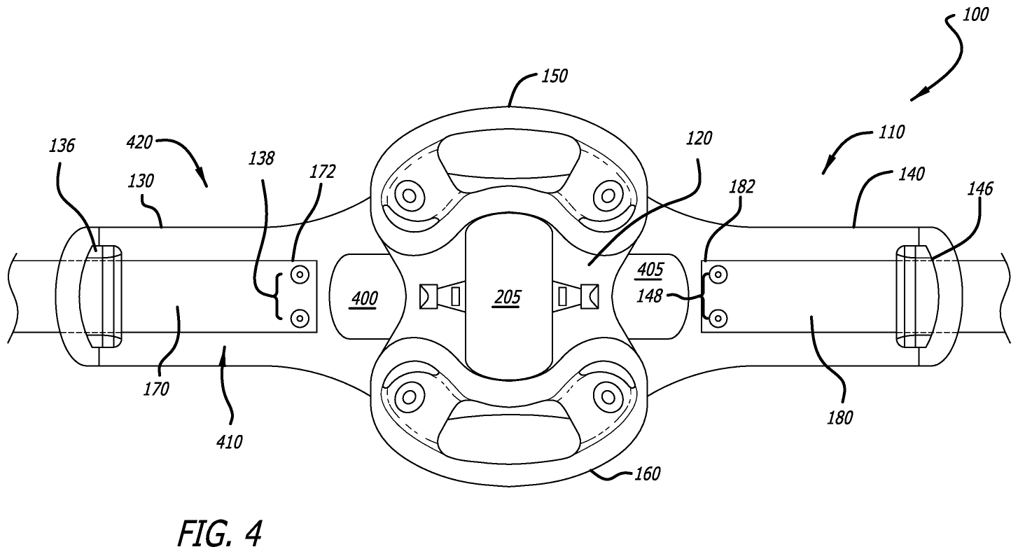

Referring now to FIG. 4, an anterior view of the cervical collar back panel 100 of FIGS. 1 & 3 is shown. The cervical collar back panel 100 features the first cervical panel 150 and the second cervical panel 160, both of which are coupled to the central panel member 120. The attachment panel 110 features aperture 205, 400, and 405, which provides the extension members 130 and 140 with greater flexibility and lateral movement when straps 170 and 180 are adjusted to connect their distal ends to the front cervical collar body as shown in FIG. 12. According to this embodiment of the disclosure, the end 172 of the first strap 170 is coupled to the first extension member 130 at the fasteners 138 and inserted through the strap slot 136. As a result, the first strap 170 propagates from an anterior surface 410 of the attachment panel 110 to a posterior surface 420 of the attachment panel 110. Likewise, the end 182 of the second strap 180 is coupled to the second extension member 140 at the fasteners and inserted through the strap slot 146 so that the second strap 180 also propagates from the anterior surface 410 of the attachment panel 110 to the posterior surface 420 of the attachment panel 110.

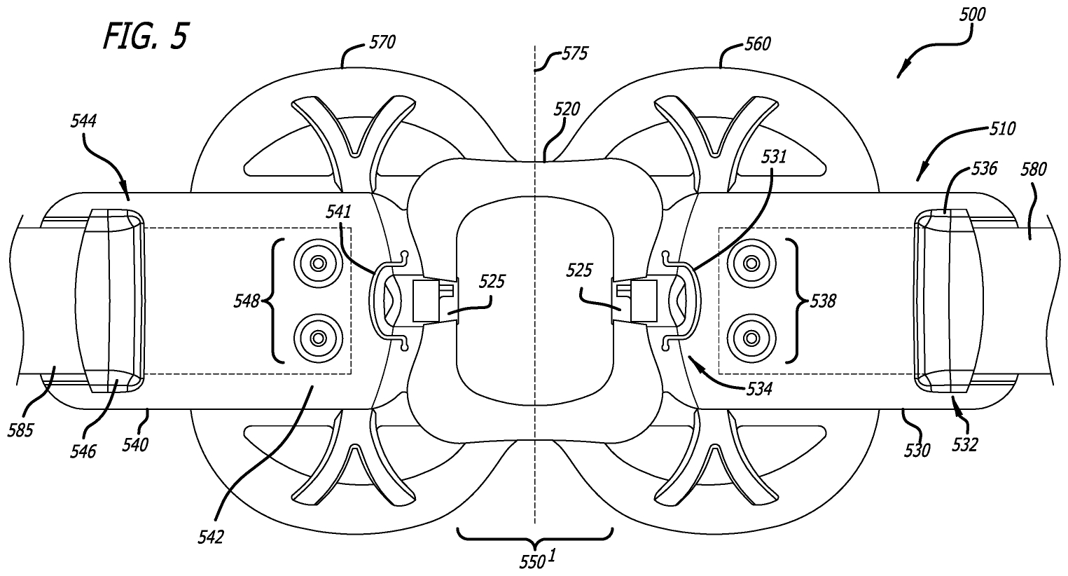

Referring to FIG. 5, a posterior view of a second exemplary embodiment of a cervical collar back panel 500 is shown. According to this embodiment, the cervical collar back panel 500 includes an attachment panel 510 and a cervical panel 550. As shown, the attachment panel 510 includes a central panel member 520, a first extension member 530 and a second extension member 540. The central panel member 520 is positioned between the first extension member 530 and the second extension member 540, where the cervical panel 550 may be coupled to the attachment panel 510 by fasteners (e.g., rivets) inserted into female fasteners 538 and 548 positioned in the first extension member 530 and the second extension member 540, respectively. While the central panel member 520, a first extension member 530 and a second extension member 540 are illustrated as a single integrated (molded) component, it is contemplated that these members 520, 530 and 540 may be separate components attached by a hinge co-located at the central panel member 520 and proximal ends of the first extension member 530 and the second extension member 540. Also, while the attachment panel 510 and the cervical panel 550 are illustrated as separate components, as another embodiment, the attachment panel 510 and the cervical panel 550 may be constructed as a single integrated component.

The central panel member 520 features inserts 525 for attachment to a spinal stabilization rod, such as the wishbone coupler of a Cervical Thoracic Lumbar Sacral Orthosis (CTLSO) described in U.S. patent application Ser. No. 18/162,367 filed Jan. 31, 2023 and entitled “Orthopedic Brace Having Telescopic Lateral Panels and an Interchangeable Wishbone Coupler,” the contents of which are incorporated by reference herein. The inserts 525 are positioned on lateral periphery members of the central panel member 520.

As further shown in FIG. 5, the first extension panel member 530 includes a proximal region 534 and a distal region 532. The proximal region 534 is coupled to the central panel member 520 while the distal region 532 extends from the central panel member 520 and features a slot 536 to receive a strap 580, which is re-located from the anterior side of the attachment panel 510 to the posterior side, for subsequent coupling to the front cervical collar body 1200 (see FIG. 12). Fasteners (e.g., rivets) are inserted into the female fasteners 538 and 548 and attached thereto, where this fastener combination is used to attach the cervical panel 550 as well as a distal end of the strap 580. The second extension panel member 540 features an architecture similar to the first extension panel member 530, where the slot 546 located in a distal region 542 of the second extension member 540 receives a strap 585 while the female fasteners 548 are located in a proximal region 544 of the second extension member 540. Herein, the straps 580/585 are different straps, although it is contemplated that the straps 580/585 may be associated with a single strap in lieu of separate straps.

Additionally, the first and second extension panel members 530 and 540 further include channels 531 and 541, respectively. The first channel 531 allows the first extension panel member 530 to flex (bend) anteriorly or posteriorly, while a spinal stabilization rod attached via the inserts 525 would remain aligned. The second channel 541 allows the second extension panel member 540 to flex anteriorly or posteriorly, where the spinal stabilization rod attached via the inserts 525 would remain aligned. Hence, the first and second channels 531 and 541 substantially mitigate or eliminate deviation from the central axis 575 due to flexing of the first and second extension panel members 530 and 540.

The cervical panel 550 includes a first segment 560 and a second segment 570. The first segment 560 is positioned laterally from a center axis 575, which is substantially in alignment with the cervical spine of the patient when the cervical collar back panel 500 is worn. The second segment 570 is positioned laterally from the center axis 575 and on an opposite side of the center axis 575 than the first segment 560.

Based on this architecture, when the cervical collar back panel 500 is installed and operating as part of a cervical collar, the segments 560 and 570 of the cervical collar back panel 500 features provide a symmetric architecture. Stated differently, the cervical panel 550 features segments 560/570 that are positioned to allow one of these segments 560 or 570 to engage with the first lateral portion of the occipital region of the patient and the first lateral portion of the upper trapezius region of the patient. The other one of these segments 570 or 560 is configured to engage with the second lateral portion of the occipital region of the patient and the second lateral portion of the upper trapezius region of the patient, where the first and second lateral portions are positioned on different sides of the cervical spine.

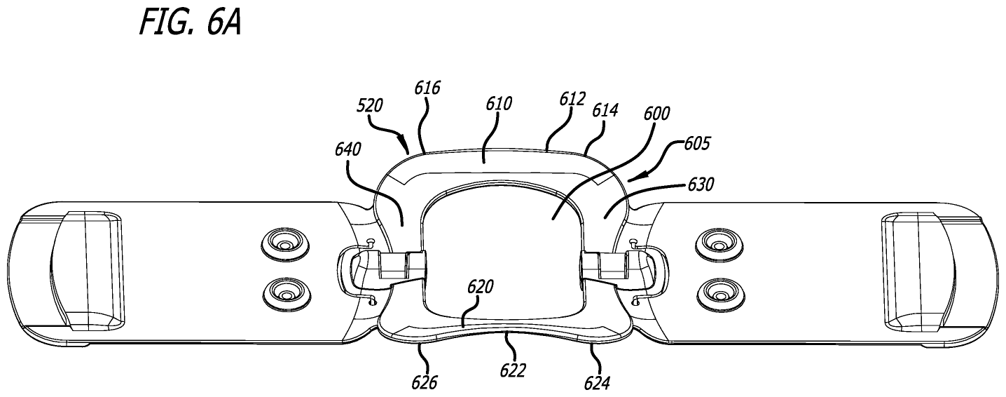

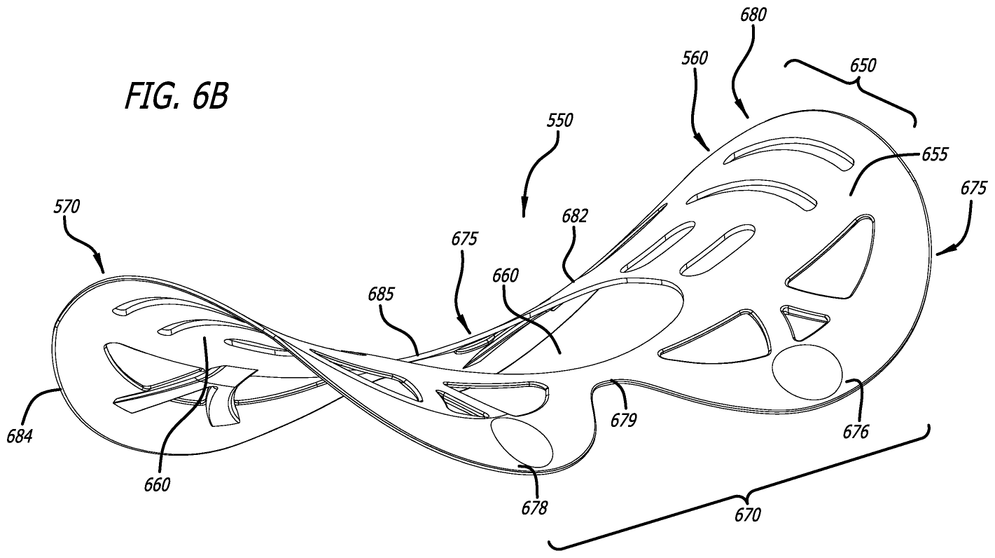

Referring now to FIG. 6A, an illustrative embodiment of the attachment panel 510 of FIG. 5 is shown. According to this embodiment, the central panel member 520 features a centralized aperture 600 formed by a plurality of periphery members 605 that are constructed with contours considering the lordotic curve of the cervical spine. For this embodiment, the plurality of periphery members 605 include a first periphery member 610, a second periphery member 620, a third periphery member 630, and a fourth periphery member 640. Herein, from a posterior viewpoint, the first periphery member 520 is configured with a concave (e.g., upward sloping) surface from the aperture 600 to an edge 612 of the first periphery member 610 while the edge 612 features a convex surface between its first distal end 614 and second distal end 616. The convex surface of the edge 612 positions the central panel member 520 away from the skin of the user for greater comfort when worn. The second periphery member 620 is configured with a concave surface from the aperture 600 to its edge 622. The edge 622 features a convex surface between its first distal end 624 and second distal end 626.

Referring still to FIGS. 6A, the third periphery member 630 is configured with a concave surface from the edge 612 of the first periphery member 610 to the edge 622 of the second periphery member 620. This concave surface is an aggregate of the concave surfaces of the first/second periphery members 610/620 from the aperture 600. Similarly, the fourth periphery member 640 is configured with the concave surface from the edge 612 of the first periphery member 610 to the edge 622 of the second periphery member 620.

In summary, with respect to the configuration of the periphery members 605, the first periphery member 610 is configured with an incline angle (e.g., upward slope) tending to veer away from an anterior side of the cervical collar back panel 500 while the second periphery member 620 is configured with a decline angle veering away from an anterior side of the cervical collar back panel 500. As a result, the first and second periphery members 610/620 are arranged to align with the lordotic curve of the cervical spine while the first and second segments 560/570 of the cervical panel 550 are arranged to align with both the occipital and upper trapezius regions of the patient.

Referring to FIG. 6B, an illustrative embodiment of the cervical panel 550 of FIG. 5. is shown. According to this embodiment, the cervical panel 550 includes a centralized lateral region 650 and a longitudinal region 670. Herein, the centralized lateral region 650 is constructed with a concave-shaped architecture, extending from an aperture 690 of the cervical panel 550 to both an edge of a lateral periphery region 655 of the first segment 560 and an edge of a lateral periphery region 660 of the second segment 570. Additionally, the longitudinal region 670 is constructed with a dual, convex-shaped architecture, extending from the aperture 690 to both a first longitudinal periphery member 675 and a second longitudinal periphery member 680. The separate convexity at edges (i.e., the first and second longitudinal periphery members 675 and 680) creates unique rigidity to retain a three-dimensional cupping shape when wrapped around the neck due to interaction of compound shapes, openings and reinforcement members illustrated herein.

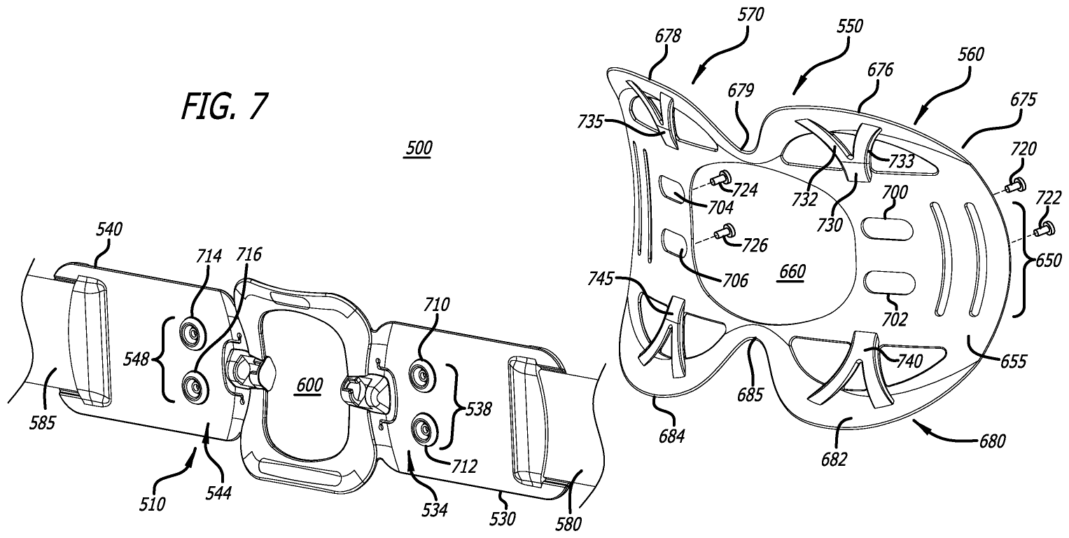

According to one embodiment of the disclosure, the first longitudinal periphery member 675 includes a first periphery member 676 being part of the first segment 560 and a second periphery member 678 being part of the second segment 570. The first longitudinal periphery member 675 further includes a recessed periphery member 679 interposed between the first periphery member 676 and the second periphery member 678. Similarly, as shown in FIGS. 6B-7, the second longitudinal periphery member 680 includes a first periphery member 682 being part of the first segment 560 and a second periphery member 684 being part of the second segment 570. The second longitudinal periphery member 680 further includes a recessed periphery member 685 interposed between the first periphery member 682 and the second periphery member 684.

Referring to FIG. 7, an exploded view of the cervical collar back panel 500 of FIG. 5 is shown. The cervical collar back panel 500 includes the attachment panel 510 and the cervical panel 550. According to this embodiment of the disclosure, the attachment panel 510 includes the female fasteners 538 positioned at the proximal region 534 of the first extension panel 530 while the female fasteners 548 are positioned at the proximal region 544 of the second extension panel member 540. As shown, a first slot 700 of the first segment 560 of the cervical panel 550, which is formed as part of the lateral periphery region 655, is aligned with a first female fastener 710 of the female fasteners 538 to receive a fastener 720 (e.g., male fastener such as a rivet, screw, bolt, etc.). The fastener 720 is partially inserted through the first slot 700 and secured by the first female fastener 710. Similarly, a second slot 702 of the first segment 560 is also formed as part of the lateral periphery region 655. The second slot 702 is aligned with a second female fastener 712 of the female fasteners 538 to receive a (male) fastener 722. The fastener 722 is partially inserted through the second slot 702 and secured by the second female fastener 712.

As further shown in FIG. 7, formed as part of the lateral periphery region 660, a third slot 704 of the second segment 570 of the cervical panel 550 is aligned with a third female fastener 714 of the female fasteners 548 to receive a fastener 724 (e.g., a male fastener such as a rivet). The fastener 724 is partially inserted through the third slot 704 and secured by the third female fastener 714. A fourth slot 706 of the second segment 570 is also formed as part of the lateral periphery region 660. The fourth slot 706 is aligned with a fourth female fastener 716 to receive a fastener 726. The fastener 726 is partially inserted through the fourth slot 706 and secured by the fourth female fastener 716.

Herein, the slots 700, 702, 704 and 706 are configured to pass through a first portion of the fasteners 720, 722, 724 and 726 and preclude a second portion of these fasteners from passing. This arrangement allows for slight lateral movement of the cervical panel 550 in relation to the attachment panel 510 in order to account for adjustments or articulations that may be needed due to physical differences between patients as well as inward compression of the segments 560/570 caused by anteriorly directed movement of the extension panel members 530/540 respectively.

As further shown in FIG. 7, reinforcement structures 730, 735, 740 and 745 are positioned between the centralized lateral region 650 and the first and second longitudinal periphery regions 675 and 680 of the cervical panel 550. As shown, according to one embodiment of the disclosure, a first reinforcement member 730 includes a first strut member 732 and a second strut member 733 that diverge to form a V-shaped structure. The first reinforcement structure 730 is attached to both the lateral periphery region 655 of the first segment 560 and the first periphery member 676 of the first longitudinal periphery member 675. The first reinforcement structure 730 is designed with rigidity to provide stability against applied forces (e.g., hold up head at occipital region, gravitational forces when applied against the upper trapezius region of the patient, etc.), while at the same time, provide some flexibility for comfort and wearability.

Similar in construction, the second reinforcement structure 735 is attached to both the lateral periphery region 660 of the second segment 560 and the second periphery member 678 of the first longitudinal periphery member 675. The third reinforcement structure 740 is attached to both the lateral periphery region 655 of the first segment 560 and the first periphery member 682 of the second longitudinal periphery member 680, while the fourth reinforcement structure 745 is attached to both the lateral periphery region 660 of the second segment 570 and the second periphery member 684 of the second longitudinal periphery member 680.

During attachment, the aperture 600 of the attachment panel 510 is aligned with the aperture 660 of the cervical panel 550. When the strap 580 is attached to the front cervical collar body, the first segment 560 is compressed inwardly. For this embodiment, the first periphery member 676 of the first longitudinal periphery member 675 would come into contact and support the occipital region of the patient. Additionally, the first periphery member 682 of the second longitudinal periphery member 680 would come into contact and receive support from the upper trapezius region of the patient. Likewise, when the strap 585 is attached to the front cervical collar body, the second segment 570 is compressed inwardly toward the first segment 560. For this embodiment, the second periphery member 678 of the first longitudinal periphery member 675 would come into contact and support the occipital region of the patient. Additionally, the second periphery member 684 of the second longitudinal periphery member 680 would come into contact and receive support from the upper trapezius region of the patient.

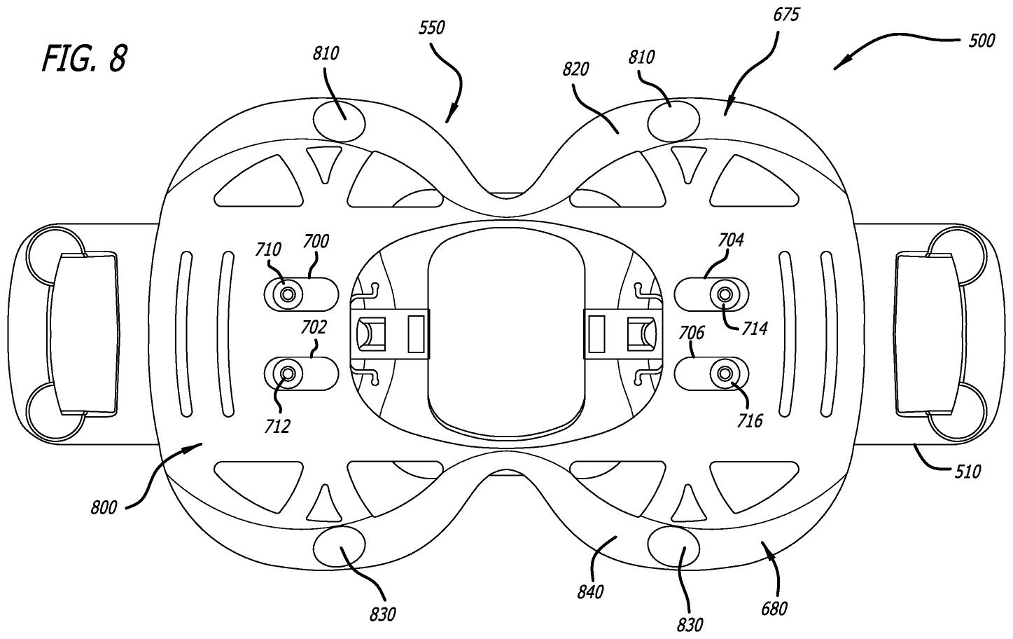

Referring to FIG. 8, an anterior view of an exemplary embodiment of the cervical collar back panel 500 of FIGS. 5 & 7 is shown. Herein, the cervical panel 550 is coupled to the attachment panel 510 from fasteners (not shown) inserted through slots 700, 702, 704 and 706 for coupling to female connectors 710, 712, 714 and 716, respectively. Adhesive members may be positioned along an interior surface 800 of the cervical panel 550. For example, a first set (e.g., one or more) adhesive members 810, such as Velcro® pads for example, may be positioned against (or attached to) an inner surface 820 of the first longitudinal periphery member 675. Additionally, or in the alternatively, a second set of adhesive members 830 may be positioned against (or attached to) an inner surface 840 of the second longitudinal periphery member 680. These adhesive members 810 and 830 are to secure padding (not shown) that is positioned over the cervical panel 550 and adjusts based on lateral movement of the segments 560 and 570 caused by affixing the straps 580 and 585 to the front cervical collar body. Although not shown, the padding features slits where the straps penetrate, which allows the padding to be changed without the need to unfeed the straps form the extension panel members.

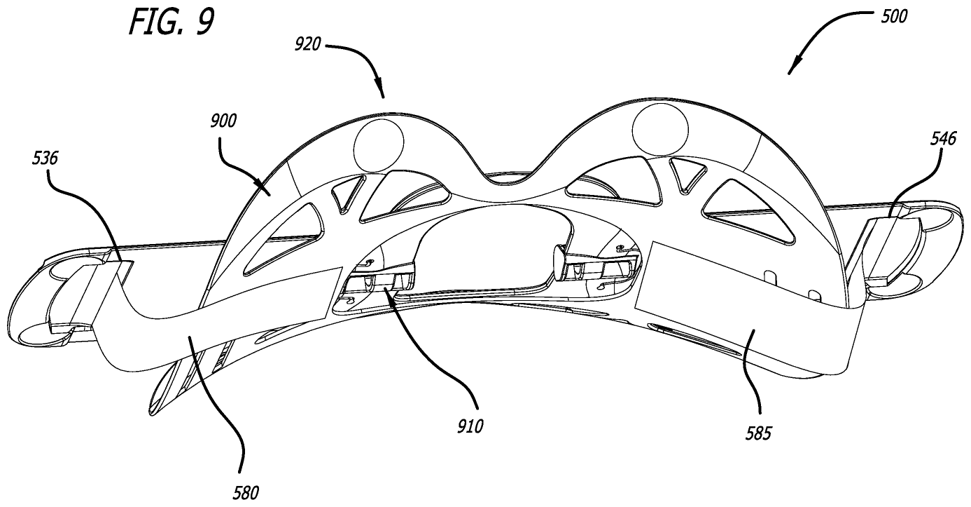

Referring to FIG. 9, an illustrative embodiment of a curvature of the cervical collar back panel 500 of FIGS. 5 & 7-8 is shown. From an anterior perspective, the cervical collar back panel 500 is configured with a concave curvature. An anterior surface 900 of the cervical panel 550 is in contact with the patient when the cervical collar back panel 500 is worn. The straps 580 and 585 are attached to an anterior side 910 of the attachment panel 510. A distal end of the strap 580 is inserted through the slot 536 for re-location on a posterior side 920 of the attachment panel 510. Similarly, a distal end of the strap 585 is inserted through the slot 546 for re-location on the posterior side 920 of the attachment panel 510.

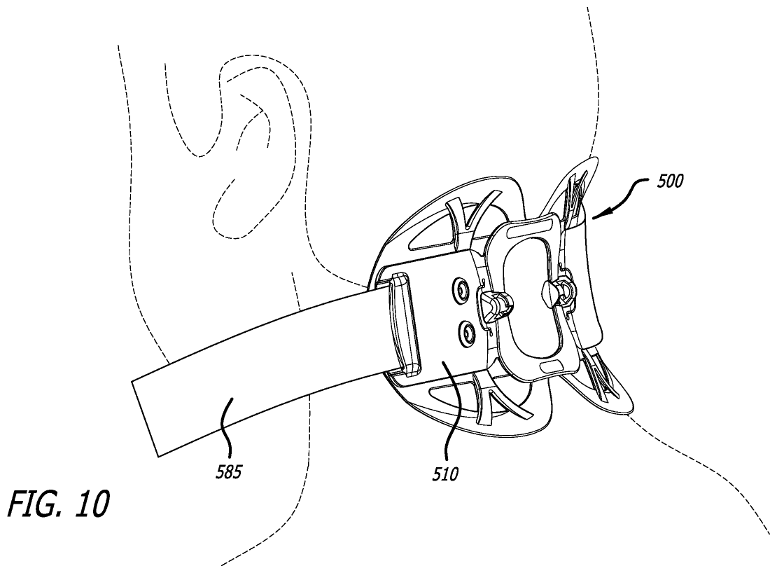

Referring now to FIGS. 10-12, an illustrative embodiment of a donning process for the cervical collar back panel 500 is shown. The cervical collar back panel 500 is positioned on the back of the neck between the occipital region and upper trapezius region of the patient as shown in FIG. 10. The strap 585 attached to the second extension member 540 of the attachment panel 510 is to be wrapped around a lateral side of a patient's neck for attachment to a front collar body 1200 of the cervical collar as shown in FIGS. 10 & 12. The distal end 1210 of the strap 585 includes a fastener 1220 for attachment to a complementary fastener 1230 situated on the front collar body 1200. As shown, the fasteners 1220/1230 may be hook & loop fasteners.

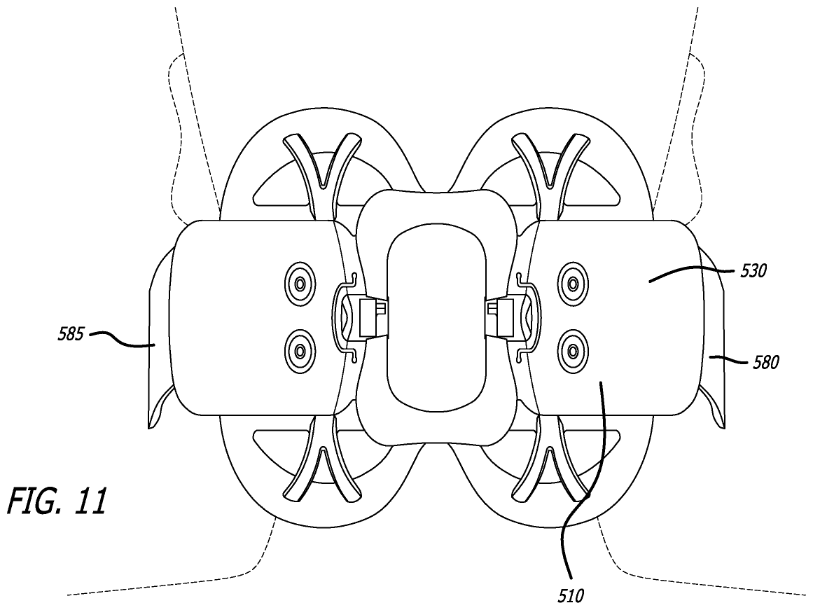

Additionally, the strap 580 attached to the first extension member 530 of the attachment panel 510 is to be wrapped around the other lateral side of a patient's neck for attachment to the front collar body 1200 of the cervical collar as shown in FIGS. 11 & 12. The distal end of the strap 580 includes a fastener (not shown) for attachment to a complementary fastener also situated on the front collar body 1200. This allows the cervical collar back panel 500 to fit snugly and be fully supported between the occipital and upper trapezius regions of the patient.

In the foregoing description, the invention is described with reference to specific exemplary embodiments thereof. However, it will be evident that various modifications and changes may be made thereto without departing from the broader spirit and scope of the invention as set forth in the appended claims.

What is claimed is:

1. A cervical collar back panel, comprising:
an attachment panel including a central panel member, a first extension panel member and a second extension panel member; and
one or more cervical panels arranged to provide a symmetric architecture during a donning process, the one or more cervical panels include (i) a first cervical panel and a second cervical panel are oriented on opposite periphery members of the central panel member or (ii) a first segment of a cervical panel and a second segment of the cervical panel, wherein the first segment of the cervical panel being configured to move laterally inward during adjustment of the first extension panel in an anterior direction and the second segment of the cervical panel being configured to move laterally inward during adjustment of the second extension panel in the anterior direction.

2. The cervical collar back panel of claim 1, wherein the symmetric architecture comprises the first cervical panel is oriented in a mirrored configuration with the second cervical panel so that the cervical collar back panel is wearable in a first orientation or a second orientation rotated one-hundred and eighty degrees From the first orientation.

3. The cervical collar back panel of claim 2, wherein the symmetric architecture comprises the first segment being oriented laterally from and in a mirrored configuration with the second segment so that the cervical collar back panel is wearable in a first orientation or a second orientation rotated one-hundred and eighty degrees From the first orientation.

4. The cervical collar back panel of claim 1, wherein the first extension panel member includes a first female fastener and a first slot located at a distal region of the first extension panel member and the second extension panel member includes a second female fastener and a second slot located at a distal region of the second extension panel member.

5. The cervical collar back panel of claim 4 further comprising:
a first strap attached to the first female fastener and residing along an anterior side of the first extension panel member, the first strap is configured to pass through the first slot to extend along a posterior side of the first extension panel member; and
a second strap attached to the second female fastener and residing along an anterior side of the second extension panel member, the second strap is configured to pass through the second slot to extend along a posterior side of the second extension panel member,
wherein the first segment is coupled to the anterior side of the first extension panel member using the first female fastener and the second segment is coupled to the anterior side of the second extension panel member using the second female fastener.

6. The cervical collar back panel of claim 1, wherein the first segment is attached to the first extension panel member to allow for inward articulation in response to compression caused by the first extension panel member and the second segment is attached to the second extension panel member to allow for inward articulation in response to compression caused by the second extension panel member.

7. The cervical collar back panel of claim 1, wherein the first extension panel member includes a channel positioned adjacent to a side periphery member of the central panel member, the channel is configured to mitigate lateral movement of the central panel member from in response to angular movement by first extension panel member.

8. The cervical collar back panel of claim 1, wherein the central panel member includes an aperture surrounding by a plurality of periphery members, the first cervical panel is attached to an anterior surface of a first periphery member of the plurality of periphery members with a first offset distance from to allow for articulation, and the second cervical panel is attached to an anterior surface of a second periphery member of the plurality of periphery members on an opposite side of the aperture from the first periphery member with a second offset distance from to allow for articulation.

9. The cervical collar back panel of claim 8, wherein, when coupled to a cervical collar front body and collectively worn as a cervical collar, the first cervical panel and the second cervical panels are interchangeable as both of the first cervical panel and the second cervical panel is configured to support either an occipital region or an upper trapezius region of a patient.

10. The cervical collar back panel of claim 1 being wearable in any of a first orientation in which the first cervical panel situated above the second cervical panel and a second orientation that is rotated one-hundred and eighty degrees From the first orientation in which the second cervical panel is situated above the first cervical panel.

11. The cervical collar back panel of claim 1, wherein the central panel member includes an aperture surrounding by a plurality of periphery members, the plurality of periphery members includes a first periphery member configured with an incline angle tending to veer away from an anterior side of the cervical collar back panel and a second periphery member, positioned on an opposite side of the aperture as the first periphery member, configured with a decline angle veering away from the anterior side of the cervical collar back panel.

12. A cervical collar back panel, comprising:
an attachment panel including a central panel member, a first extension panel member and a second extension panel member; and
a cervical panel including a first segment and a second segment, the first segment being oriented symmetrically in a lateral and a mirrored configuration with the second segment so that the cervical collar back panel is wearable in a first orientation or a second orientation rotated one-hundred and eighty degrees From the first orientation.

13. The cervical collar back panel of claim 12, wherein the first segment of the cervical panel being configured to move laterally inward during adjustment of the first extension panel in an anterior direction and the second segment of the cervical panel being configured to move laterally inward during adjustment of the second extension panel in the anterior direction.

14. The cervical collar back panel of claim 11, wherein the first extension panel member includes a first female fastener and a first slot located at a distal region of the first extension panel member and the second extension panel member includes a second female fastener and a second slot located at a distal region of the second extension panel member.

15. The cervical collar back panel of claim 14 further comprising:
a first strap attached to the first female fastener and residing along an anterior side of the first extension panel member, the first strap is configured to pass through the first slot to extend along a posterior side of the first extension panel member; and
a second strap attached to the second female fastener and residing along an anterior side of the second extension panel member, the second strap is configured to pass through the second slot to extend along a posterior side of the second extension panel member,
wherein the first segment is coupled to the anterior side of the first extension panel member using the first female fastener and the second segment is coupled to the anterior side of the second extension panel member using the second female fastener.

16. The cervical collar back panel of claim 12, wherein the first segment is attached to the first extension panel member to allow for inward articulation in response to compression caused by the first extension panel member and the second segment is attached to the second extension panel member to allow for inward articulation in response to compression caused by the second extension panel member.

17. The cervical collar back panel of claim 12, wherein the first extension panel member includes a channel positioned adjacent to a side periphery member of the central panel member, the channel is configured to mitigate lateral movement of the central panel member from in response to angular movement by first extension panel member.

18. The cervical collar back panel of claim 12, wherein, when coupled to a cervical collar front body and collectively worn as a cervical collar, the first segment and the second segment are interchangeable as both the first segment and the second segment are configured to support different occipital region and upper trapezius regions of a patient.

19. The cervical collar back panel of claim 12, wherein the cervical panel is configured to be oriented in one of a first orientation and a second orientation, the first orientation including the first segment laterally offset in a first lateral direction from a central axis corresponding to an intended location of a cervical spine of a patient and the second segment laterally offset in a second lateral direction opposite from the first lateral direction from the central axis, and the second orientation including the second segment laterally offset in the first lateral direction from the central axis and the first segment laterally offset in the second lateral direction from the central axis.

20. The cervical collar back panel of claim 12, wherein the central panel member includes an aperture surrounding by a plurality of periphery members, the plurality of periphery members includes a first periphery member configured with an incline angle veering away from an anterior side of the cervical collar back panel and a second periphery member, positioned on an opposite side of the aperture as the first periphery member, configured with a decline angle veering away from the anterior side of the cervical collar back panel.

21. A cervical collar, comprising:
a front collar body; and
a cervical collar back panel coupled to the front collar body, the cervical collar back panel comprises
an attachment panel including a central panel member, a first extension panel member and a second extension panel member, and
a cervical panel including a first segment and a second segment, the first segment being oriented symmetrically in a lateral and a mirrored configuration with the second segment so that the cervical collar back panel is wearable in a first orientation or a second orientation rotated one-hundred and eighty degrees From the first orientation.
